(19) United States
(12) Patent Application Publication
(10) Pub. No.: US 2025/0120841 A1
(43) Pub. Date: Apr. 17, 2025

(54) FINGER SPLINT

(71) Applicant: Safe Splints, LLC, Winter Park, FL (US)

(72) Inventor: Jeffrey A. Deren, Winter Park, FL (US)

(73) Assignee: Safe Splints, LLC, Winter Park, FL (US)

(21) Appl. No.: 18/912,881

(22) Filed: Oct. 11, 2024

Related U.S. Application Data
(60) Provisional application No. 63/543,630, filed on Oct. 11, 2023.

(51) Int. Cl. A61F 5/058

(52) U.S. Cl. CPC A61F 5/05875

(57) ABSTRACT

Finger splint. A finger splint referenced herein is configured to hyperextend portions of the finger while still allowing for a full range of motion of the proximal inter-phalangeal (PIP) joint. Such a splint is useful to treat a mallet finger, which is an injury to the thin tendon that straightens the end joint of a finger.

### PRIORITY

The present application is related to, and claims the priority benefit of, U.S. provisional patent application Ser. No. 63/543,630, filed Oct. 11, 2023, the contents of which are incorporated herein directly and by reference in their entirety.

### BRIEF SUMMARY

The present disclosure includes disclosure of a finger split configured to hyperextend portions of the finger while still allowing for a full range of motion of the proximal inter-phalangeal (PIP) joint. Such a splint is useful to treat a mallet finger, which is an injury to the thin tendon that straightens the end joint of a finger.

The present disclosure includes disclosure of a splint device, comprising a flexible ring or cuff, and an extension portion extending from the flexible ring or cuff, the extension portion configured to maintain hyperextension of a distal interphalangeal (DIP) joint when the splint device is worn about a finger. By hyperextending the DIP joint and keeping the same in a hyperextended period for a duration of time, such as 6-8 weeks for example, this allows the tendon to reconnect and heal so to treat the mallet finger.

The present disclosure includes disclosure of a splint device, wherein the flexible ring or cuff is configured to fit around the finger at or proximal to the proximal inter-phalangeal (PIP) joint.

The present disclosure includes disclosure of a splint device, wherein the flexible ring or cuff is configured to grip the finger at or proximal to the PIP joint and not slide.

The present disclosure includes disclosure of a splint device, wherein the flexible ring or cuff comprises a material selected from the group consisting of silicone, rubber, and soft plastic.

The present disclosure includes disclosure of a splint device, wherein the extension portion comprises a plastic splint.

The present disclosure includes disclosure of a splint device, wherein the extension portion comprises a tape or ribbon.

The present disclosure includes disclosure of a splint device, wherein the tape or ribbon is connected to the ring or cuff at a bottom portion of the ring or cuff, extends around the tip of the finger, and attaches to a top portion of the ring or cuff using a connector.

The present disclosure includes disclosure of a splint device, wherein the connector allows tension of the tape or ribbon to be adjusted so to maintain hyperextension of the DIP joint but not cause discomfort to the user.

The present disclosure includes disclosure of a splint device, further comprising rigid dorsal splint configured to be located on a dorsal side of the finger at the DIP joint.

The present disclosure includes disclosure of a splint device, as shown and/or described herein.

The present disclosure includes disclosure of a method of treating mallet finger, as shown and/or described herein.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a splint device positioned about a finger without flexing the proximal inter-phalangeal (PIP) joint, according to an exemplary embodiment of the present disclosure;

FIG. 2 shows a splint device positioned about a finger without flexing the PIP joint, according to an exemplary embodiment of the present disclosure;

FIG. 3 shows a splint device positioned about a finger while flexing the PIP joint, according to an exemplary embodiment of the present disclosure;

FIG. 4 shows a splint device positioned about a finger without flexing the PIP joint, according to an exemplary embodiment of the present disclosure;

FIG. 5 shows a splint device positioned about a finger while flexing the PIP joint, according to an exemplary embodiment of the present disclosure;

FIG. 6 shows a splint device positioned about a finger without flexing the PIP joint, according to an exemplary embodiment of the present disclosure;

FIG. 7 shows a splint device positioned about a finger while flexing the PIP joint, according to an exemplary embodiment of the present disclosure; and

FIG. 8 shows a splint device positioned about a finger without flexing the PIP joint, according to an exemplary embodiment of the present disclosure.

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

The present disclosure includes disclosure of a finger split configured to hyperextend portions of the finger while still allowing for a full range of motion of the proximal inter-phalangeal (PIP) joint.

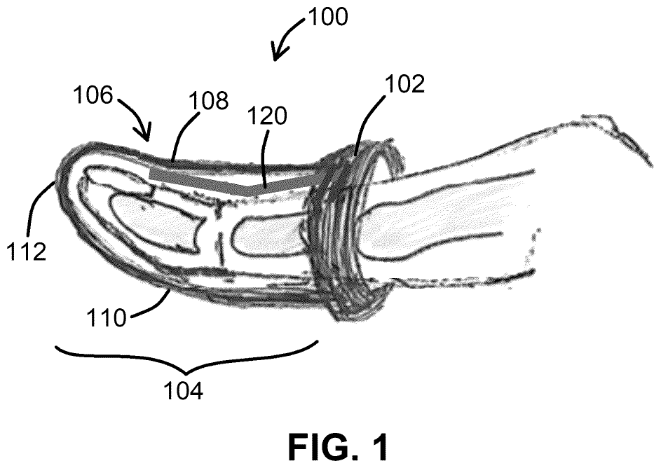

An exemplary splint device 100 of the present disclosure is shown in FIG. 1. As shown therein, device 100 comprises a flexible ring or cuff 102 configured to fit around a user's finger at or proximal to the PIP joint. Flexible ring or cuff 102 may comprise any number of comfortable materials, such as silicone, rubber, soft plastic, or another type of flexible material that can hold the splint device 100 in place while not providing any uncomfortable compressive or tight circumferential pressure around the finger. Any meaningful compressive or circumferential pressure can cause swelling and cut off blood flow, neither of which is desirable when trying to treat mallet finger. Said flexible ring or cuff 102 generally would need to grip the finger and not slide, but do so without providing uncomfortable compressive or circumferential pressure around the finger. FIG. 1 shows a splint device 100 being worn about a finger while the PIP joint is straight (not bent).

An extension portion 104 extends from flexible ring or cuff 102 as shown in FIG. 1. In at least one embodiment, the extension portion 104 is configured as a plastic splint 106 extending from flexible ring or cuff 102, whereby an upper portion of plastic splint 108 and a lower portion of plastic splint 110 are each curved along their extended length, such that the curvature generally extends upward in a manner that gently hyperextends the distal interphalangeal (DIP) joint. Upper portion 108 and lower portion 110 come together at distal portion 112, so that upper portion 108, distal portion 112, and lower portion 110 are contiguous and extending from flexible ring or cuff 102. As such, device 100 is configured to create hyperextension across the DIP joint without obstructing motion of the PIP joint. Hyperextension of the DIP joint is maintained by longitudinal forces along the finger, which is what allows the device 100 to maintain the hyperextension without compromising the blood flow or creating significant swelling or patient discomfort, which is a unique feature of the present disclosure.

In at least one embodiment, ring or cuff 102 is inflatable.

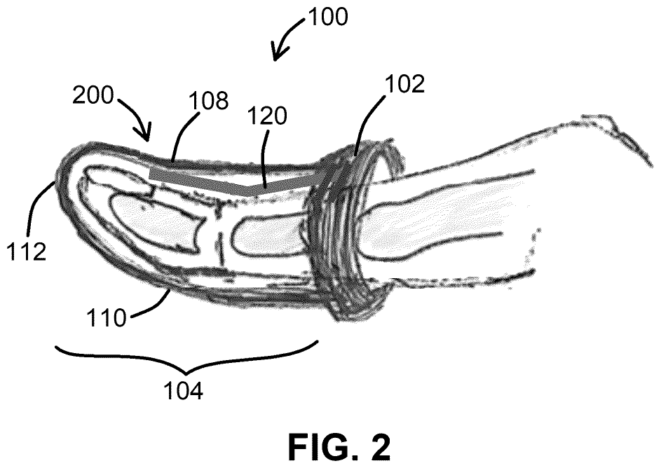

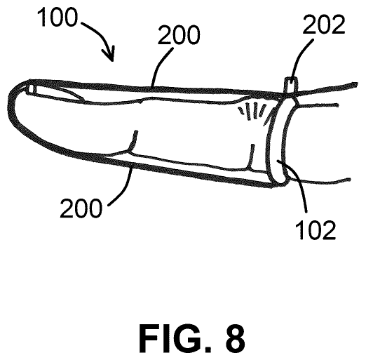

In at least another embodiment, extension portion 104 can be configured as a tape or ribbon 200, such as shown in FIG. 2, extending from flexible ring or cuff 102 and having an upper portion 108 and a lower portion 110 coming together at a distal portion 112, whereby the tension of the tape or ribbon 200 gently hyperextends the DIP joint. Such an embodiment would have the tape or ribbon 200 attached to the ring or cuff 102 on the bottom side of the ring or cuff 102, wrap around the front of the finger, and then be attached to the top side of the ring of cuff using a connector 202, such as shown in FIG. 8, that is configured to hold that portion of the tape or ribbon 200 in place. By using the connector 202, the hyperextension extent can be controlled so that the DIP joint remains hyperextended but without causing discomfort to the wearer. Connector 202 can be a post, a buckle, a clasp, or another means of retaining tape or ribbon 200 as desired.

Splint devices 100 of the present disclosure may also be referred to as “mallet splints.”

Exemplary splint devices 100 of the present disclosure are configured so to provide stabilizing pressure that is longitudinally oriented along the finger from the volar side to the dorsal side. This unique feature of splint devices 100 makes said devices operate as desired, maintaining the DIP joint in a slightly hyperextended configuration.

As shown in FIG. 1, an exemplary splint device 100 of the present disclosure can also comprise a rigid dorsal splint 120 configured to be located on the dorsal side of the finger at the DIP joint. Said rigid dorsal splint 120 would have either a gentle curvature or a V-shape, so that when extension portion 104 is positioned around a finger, said rigid dorsal splint 120 would help keep the DIP joint in a hyperextended configuration.

In at least one embodiment, and when splint device 100 is worn, splint device 100 completely surrounds the portion of the finger at the DIP joint. In other embodiments, and when splint device 100 is worn, said device does not completely surround the portion of the finger at the DIP joint.

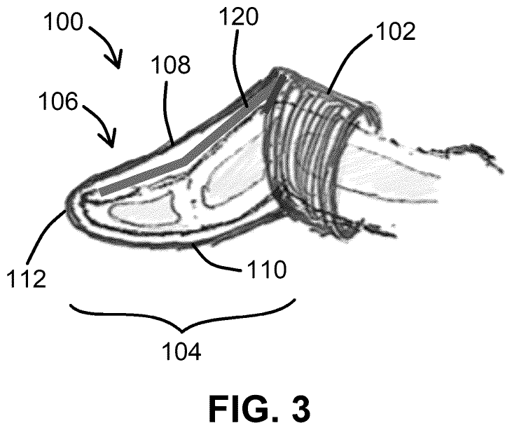

FIG. 3 shows an embodiment of a splint device 100 positioned about a finger with the PIP joint in a bent configuration. Said figure shows that the splint device 100 remains in place, and the DIP joint remains in a hyperextended position, even when the PIP joint is bent.

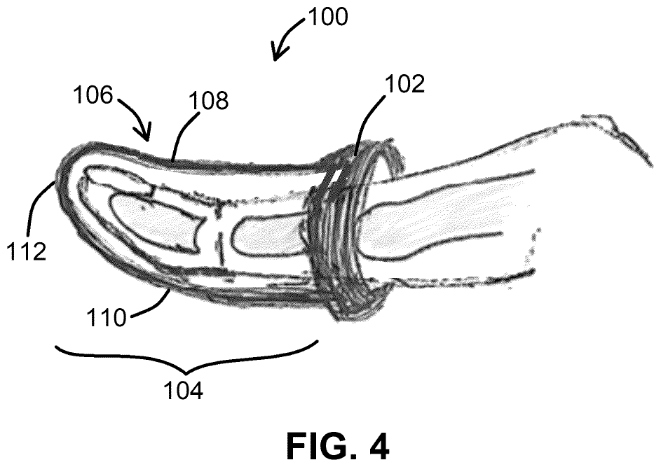

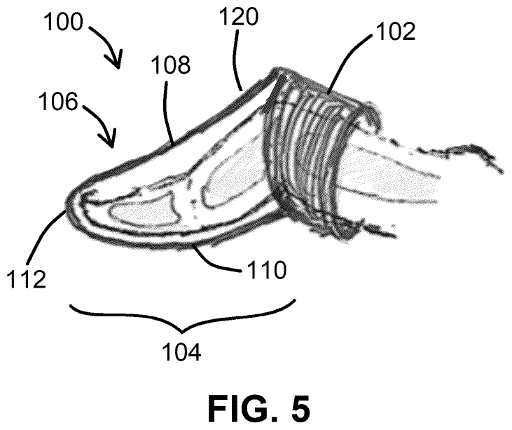

FIGS. 4 and 5 show an embodiment of a splint device 100 of the present disclosure, such as shown in FIG. 1, but without a rigid dorsal splint 120 comprising part of said splint device.

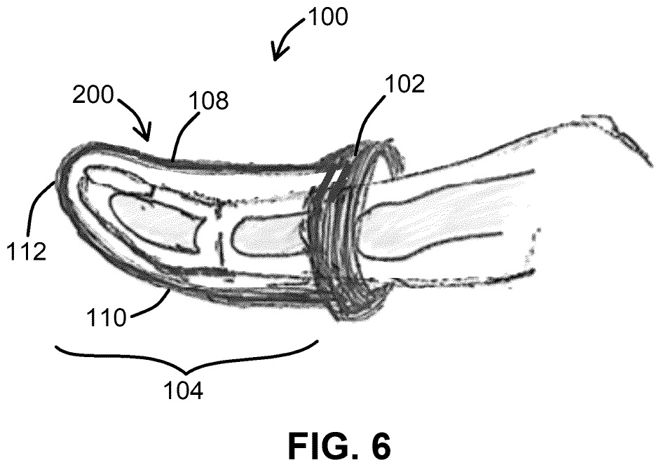

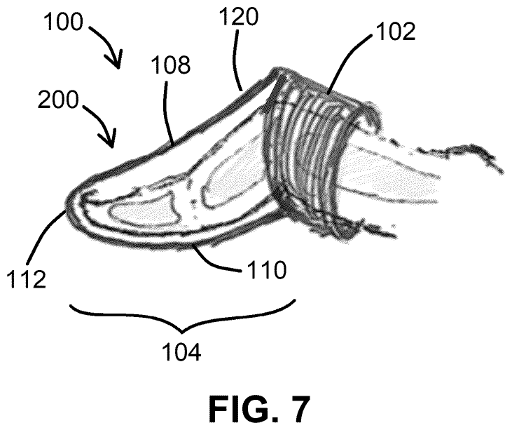

FIGS. 6 and 7 show an embodiment of a splint device 100 of the present disclosure, such as shown in FIG. 2, but without a rigid dorsal splint 120 comprising part of said splint device.

FIG. 8 shows a splint device 100 of the present disclosure comprising a ring or cuff 102, a tape or ribbon 200, and a connector, positioned about and hyperextending a finger. In at least one embodiment, an adhesive can be positioned at the fingertip to hold the tape or ribbon 200 in place. In various embodiments, splint devices 100 are waterproof or water-resistant.

While various embodiments of devices for and methods for using the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A splint device, comprising:
a flexible ring or cuff; and
an extension portion extending from the flexible ring or cuff, the extension portion configured to maintain hyperextension of a distal interphalangeal (DIP) joint when the splint device is worn about a finger.

2. The splint device of claim 1, wherein the flexible ring or cuff is configured to fit around the finger at or proximal to the proximal inter-phalangeal (PIP) joint.

3. The splint device of claim 2, wherein the flexible ring or cuff is configured to grip the finger at or proximal to the PIP joint and not slide.

4. The splint device of claim 1, wherein the flexible ring or cuff comprises a material selected from the group consisting of silicone, rubber, and soft plastic.

5. The splint device of claim 1, wherein the extension portion comprises a plastic splint.

6. The splint device of claim 1, wherein the extension portion comprises a tape or ribbon.

7. The splint device of claim 6, wherein the tape or ribbon is connected to the ring or cuff at a bottom portion of the ring or cuff, extends around the tip of the finger, and attaches to a top portion of the ring or cuff using a connector.

8. The splint device of claim 7, wherein the connector allows tension of the tape or ribbon to be adjusted so to maintain hyperextension of the DIP joint but not cause discomfort to the user.

9. The splint device of claim 1, further comprising:
rigid dorsal splint configured to be located on a dorsal side of the finger at the DIP joint.
